(19) United States
(12) Patent Application Publication
(10) Pub. No.: US 2008/0076137 A1
(43) Pub. Date: Mar. 27, 2008

(54) Bioanalytic Systems and Methods

(76) Inventors: Nicolai Bovin, Moscow (RU); Alexander Chinarev, Moscow (RU)

Correspondence Address: WILSON SONSINI GOODRICH & ROSATI, 650 PAGE MILL ROAD, PALO ALTO, CA, 94304-1050, US

(21) Appl. No.: 11/828,227

(22) Filed: Jul. 25, 2007

Related U.S. Application Data
(60) Provisional application No. 60/833,249, filed on Jul. 26, 2006.

(51) Int. Cl. G01N 33/53; C07D 495/04; C07H 15/00

(52) U.S. Cl. 435/7.5; 536/1.11; 548/303.7

(57) ABSTRACT

Glycopolyiners configured for single point binding to a substrate and methods of use thereof are provided.

### CROSS-REFERENCE

This application claims the benefit of U.S. Provisional Application No. 60/833,249, filed Jul. 26, 2006, which application is incorporated herein by reference.

### FIELD OF THE INVENTION

The invention relates to carbohydrate-containing molecules that are utilized in bioanalytical systems, biosensing methods and business methods related thereto. In an exemplary embodiment, glycopolymers are carried in an array, on beads or in a microfluidic system for diagnostic screening for risk of neoplasia, the existence of neoplasia in a patient, or for treatment monitoring. In such an embodiment, the bioatialytic system identifies binding interactions between molecules in a patient test sample and the glycopolymers. These systems can use the glycan compositions to generate an immune response against cancer cell epitopes. Alternatively, antibody therapeutics can be developed that are useful for binding to the glycan compositions on a cell surface.

### BACKGROUND OF THE INVENTION

Cell surfaces carbohydrates, glycoproteins and glycolipids have multiple biological functions. Abnormalities in glycosylation are one of the basic mechanisms of malfunction (pathology) in living organisms, and particularly in cancers. Consequences of abnormal glycosylation are alteration of cell-cell recognition and signaling, activation of immune response, deregulation of cellular and tissue functions, and—if persisting—may result in malignant transformation. Malignant transformation and tumor progression can be correlated with specific changes in such complex surface carbohydrates, known as tumor-associated carbohydrate antigens (TACAs).

The development of nucleotide and protein microarrays has revolutionized genomic, gene expression and proteomic research. The development of glycan microarrays has been very slow for a number of reasons. First, ii has proven difficult to immobilize a library of chemically and structurally diverse glycans on arrays, beads or-the-like. Second, it is difficult to provide glycan binding to a support that optimally exposes the three-dimensional glycan structure on the array or bead surface. Thus, new glyco-compounds and linking systems are needed for advancing bioanalytic systems for early cancer detection and target discovery.

A glycan array-has been described in PCT/US2005/007370 filed Mar. 7, 2005 titled “High Throughput Glycan Microarrays” and U.S. Provisional Patent Application No. 60/629,666 filed Nov. 19, 2004 titled “Development of Blood Based Test Allowing Diagnosis of Neoplasia Status”, both of which are incorporated herein by this reference in their entirety and made a part of this specification.

### SUMMARY OF THE INVENTION

In general in one aspect of the invention a glycopolymer configured for-single point binding to a substrate is provided. In one embodiment the glycopolymer includes a configuration for single point binding that includes a single biotin molecule coupled to the glycopolymer. Furthermore, the substrate can include immobilized streptavidin or a streptavidin derivative. In a particular embodiment the biotin molecule is an end group of the glycopolymer. In one embodiment the glycopolymer includes the compound of formula 3 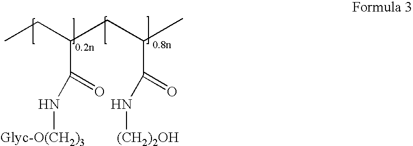
wherein n is between 30 to 10,000 and wherein Glycₓ comprises at least one of the carbohydrates listed in Table 1.

The substrate can be selected from at least one of the group including a bead, microsphere, slide, plate, stick, probe, array and a liposome. It is envisioned that the substrate can include a material selected from the group including a polymer, glass, metal and a ceramic.

In one embodiment the glycopolymer includes at least one of the carbohydrates listed in Table I. In another embodiment the glycopolymer includes a fluorescent label. In a particular embodiment the fluorescent label includes a single fluorescent label. It is envisioned that the glycopolymer can include at least one of the group including a carbohydrate-containing molecule, a macromolecule, a monosaccharide, an oligosaccharide, a polysaccharide, a glycolipid, a glycoprotein and a mimetic of a carbohydrate or carbohydrate-containing molecule.

In a related aspect the glycopolymer of the invention can be used in the monitoring, diagnosing and/or prognosing of a state of health of a subject based on a subject test sample. In one such embodiment monitoring, diagnosing and/or prognosing the state of health of the subject includes reviewing or analyzing data relating to antibodies in the subject test sample; providing a conclusion to a patient, a health care provider or a health care manager, the conclusion being based on the review or analysis of data regarding monitoring, diagnosing and/or prognosing the state of health of the subject. In a particular embodiment providing a conclusion includes transmission of the data over a network.

In another related aspect the glycopolymer of the invention is used with a flow cytometry system including a plurality of beads carrying the glycopolymer, using flow cytometry to detect a binding interaction between antibodies in the subject test sample and the glycopolymer, and utilizing an algorithm to identify a pattern of binding interactions previously identified as being associated with a neoplasia or risk of neoplasia. In one embodiment the glycopolymer includes a plurality of different glycopolymers.

In general in another aspect the invention provides a method of synthesizing a glycoconjugate including a glycopolymer and a single biotin tag by ligating the compound of formula 1 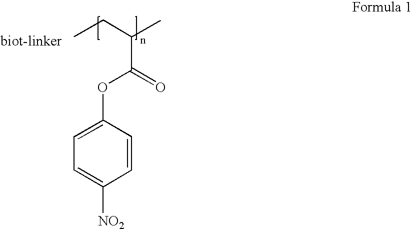
wherein n is between 30 to 10,000, with Glyc-O(CH₂)₃NH₂ (formula 2) wherein Glyc comprises at least one of the carbohydrates listed in Table 1. In one embodiment the compound of formula 1 is produced using alkycobalt(III) chelate with tridentate Schiff base coupled to biotin for initiation of polymerization of 4-nitrophenylacrylate. In a related embodiment the glycopolymer comprises at least one of the carbohydrates listed in Table 1. In another embodiment the glycopolymer further includes a single fluorescent label.

In general in another aspect the invention provides a glycopolymer comprising a single biotin group.

In general in yet another aspect the invention provides a compound of formula 1 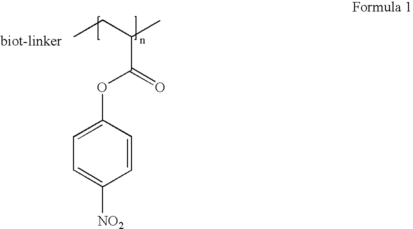
wherein n is between 30 to 10,000 and wherein biot is a single biotin.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a block diagram and flow chart illustrating aspects of a business method corresponding to the invention.

FIG. 2 is a block diagram showing a representative example of a kit.

FIG. 3 is a block diagram showing a representative example logic device in communication with an apparatus for use with the invention.

FIG. 4 illustrates (a) a synthetic approach which leads to glycopolymers with several pendant side biotins and (b) the incorporation of a biotin tag into the polymer as a fragment of initiator followed by ligation with Glyc-O(CH2)3NH2.

FIG. 5 illustrates glycolarrays constructed on Streptavidin-coated surface with glycopolymers carrying randomly pendant side biotin groups(left), glycopolymers with end biotin groups (center) or monovalent biotinylateil glycosides (right).

FIG. 6 shows the structure of biotinylated organocobalt initiator (top) and the scheme of its synthesis (bottom).

FIG. 7 depicts ¹H NMR spectrum of biot-PHEAA in D₂O.

FIG. 8 is a graph showing the affinity of antibodies to different biotinilated glycoconjugates.

### DETAILED DESCRIPTION OF THE INVENTION

The systematic study of biological processes driven by carbohydrate recognition requires multivalent carbohydrate probes. Linear polymers with pendant carbohydrates groups (Glyc; glycoside residue herein), also termed glycopolymers in this disclosure, are probably the most practical tools for this purpose (see, e.g., N. V. Bovin in Chemical Probes in Biology; (Ed. M. P. Schneider), Kluwer Academic Publishers, The Netherlands, 2003, pp. 207-225; and N. V. Bovin, Glycoconjugate J., 1998, 15, pp. 431-446, the entire contents of which are incorporated herein by this reference and made a part of this specification). Libraries of polyacrylamides with various pendant carbohydrate residues and labels, for example biotin, are currently available for functional glycomics research (see Consortium for Functional Glycomics. http://glycomics.scripps.edu (accessed March 2006)). Development of bioanalytic systems and methods utilizing carbohydrate bioanalytic systems, biosensing structures and/or carbohydrate arrays need improved methods for deposition and immobilization of such glycopolymers on the surface of an array, probe, bead or the like.

One of the approaches used for glycopolymer immobilization is based on application of a biotin-streptavidin system, when glycopolymers bearing biotin residues are anchored to a Streptavidin-coated surface. (see O. E. Galanina, M. Mecklenburg, N. E. Nifantiev, G. V. Pazynina, N. V. Bovin, Lab. Chip, 2003, 3, 260-265). This procedure affords quantitative yield of immobilization. The modified surface is covered by Glyc clusters presented for multivalent interaction with carbohydrate binding proteins, cells, pathogens and the like.

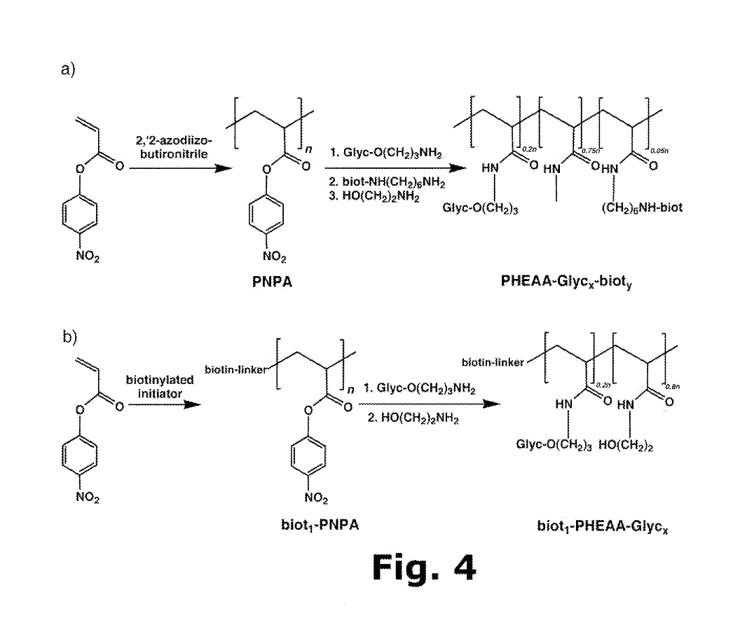

Syntheses of biotinylated glycopolymers are conventionally accomplished in accordance with the scheme shown in FIG. 4a. However, the molecules of glycopolymers synthesized in a conventional manner will contain several biotin residues, and thus be capable of multipoint binding to a Streptavidin-coated surface. As a result, flexibility of the polymer chain is reduced, and multivalent interaction is constrained. In addition, such “extra” biotins can cause non-specific interactions, especially when complex biological fluids (e.g., serum) or cells are analyzed.

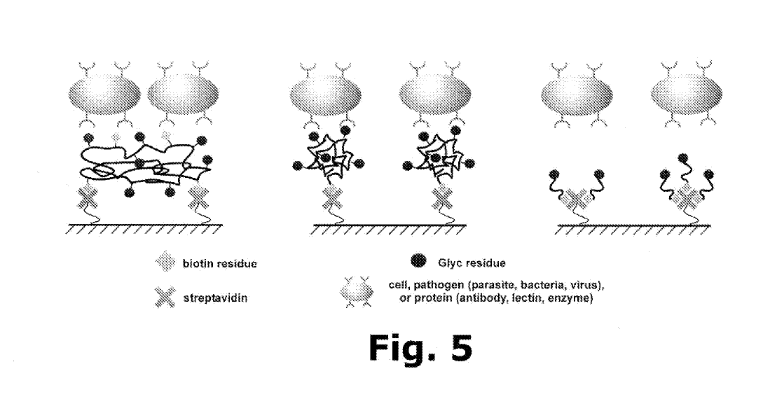

FIG. 5 illustrates an example of the scheme demonstrating glycolarrays constructed on Streptavidin-coated surface with glycopolymers carrying randomly pendant side biotin groups(left), glycopolyiners with end biotin groups (center) or monovalent biotinylated glycosides (right). In one embodiment of the invention, glycopolymers are provided that carry only one biotin residue per macromolecule which eliminates the above-described disadvantages (see FIG. 5). The same applies to biotinylated glycosides; however a large amount of a monovalent derivatives tend to make such arrays with low Glyc density to be less than optimal for realization of multivalent interactions. (see K. R. Love, P. H. Seeberger, Angew. Chem. Int. Ed., 2002, 41, 3583-3586; and O. Blixt, S. Head, T. Mondala, C. Scanlan, M. Huflejt, R. Alvarez, M. Bryan, et al. PNAS, 2004, 101, 17033-17038).

As illustrated in FIG. 4a the currently used synthetic approach leads to glycopolymers with several pendant side biotins. One embodiment of the invention as illustrated in FIG. 4b, uses the incorporation of a biotin tag into the polymer as a fragment of initiator followed by ligation with Glyc-O(CH₂)₃NH₂. Using conventional methods, it is not possible to couple exactly one biotin tag with the polymer (see FIG. 4a), which always leads to statistical distribution of biotin residues. This disclosure provides techniques and methods for providing poly(4-nitrophenylacrylate), PNPA, with a single end biotin group (see FIG. 4b).

In a particular embodiment, the glycopolymer of the invention can include the compound of formula 3 (biot₁-PHEAA-Glycₓ). 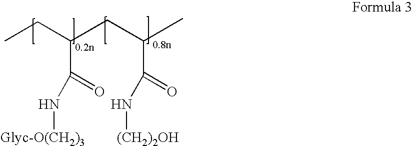
It is envisioned that n of formula 3 can be between 30 to 10,000 and Glyc can include at least one of the carbohydrates listed in Table 1 (see below). The same applies for Glyc and n in Glyc-O(CH₂)₃NH₂ (formula 2) and formula 1 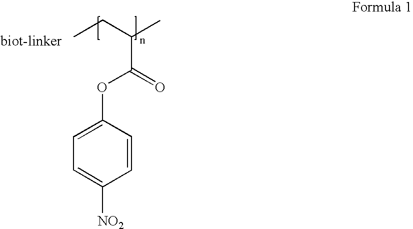
respectively. Useful compounds of formula 1 and formula 3 can include values for n ranging between 100 to 7,500; 250 to 5,000; 500 to 2,000 or 900 to 1,100.

In many cases, vectors, labels, anchors, bioligands and other entities modulating physico-chemical or biological properties may be attached to a linear polymer via its end-chain chemical groups (—OH, —COOH, —CN, —NH₂ and etc.). (see K. Huang, B. P. Lee, D. R. Ingram, P. B. Messersmith, Biomacromolecules, 2002, 3, 397-406; F. Ahmed, P. Alexandridis, S. Neelameggham, Langmuir, 2001, 17, 537-546; and K. Ulbrich, V. Subr, J. Strohalm, D. Plocova, M. Jelinkova, B. Rinova, J. Control. Release, 2000, 64, 63-79).

In one example, glycopolymers with a single fluorescent reporter group were synthesized by ruthenium carben-initiated living ring-opening metathesis polymerization (ROMP). Polymerization was terminated with a specially tailored monomer provided with a functional group, which allows post synthetic installation of the reporter group. (see R. M. Owen, J. E. Getstwicki, T. Young, L. L. Kiessling, Organic Lett., 2002,14, 2293-2296; and E. J. Gordon, J. E. Getstwicki, L. Strong, L. L. Kiessling, Chem&Biol, 2000, 7, 9-16).

Alternatively, a functional entity may be introduced into a polymer directly during its synthesis with a fragment of initiator and/or chain transfer agent. The second approach is beneficial for construction of complex heterofunctional polymers. Thus, preparation of lipid-terminated polymers with pendant side carbohydrate residues is described. For this end, C₁₈ lipid was conjugated to 4,4′-azobis(4-cyanopentanoic acid), which was used to polymerize methylvinylketon; the polymer was ligated with α-aminooxy-GalNAc moieties. Immobilization of the obtained polymeric amphiphile on surface of carbon nanotubes was reported. (see X. Cheri, S. G. Lee, A. Zettl, C. Bertozzi, Angew. Chem. Int. Ed., 2004, 43, 6112-6116). Other examples, when biofunctional polymers with long hydrophobic alkyl tails were obtained in the similar way, can be found in literature. (see M. Niwa; T. Sawada, N. Higashi, Langmuir, 1998, 14, 3916-3920; and N. D. Winblade, H. Schmokel, M. Baumann, A. S. Hoffman, J. A. Hubbell, J. Biomed. Mat. Res., 2002, 59, 618-631).

In one embodiment of the invention, synthesis of an active ester homopolymer is described with an end biotin group using a biotinylated radical initiator, and further conversion of the polymer obtained into a glycoconjugate. Among precursor-molecules that would be coupled to biotin and then used to initiate polymerization of 4-nitrophenylacrylate without unwanted side process, one embodiment selects an alkylcobalt (III) chelate with tridentate Schiff base. Such a complex can serve as a source of carbocentered radicals generated due to homolytic splitting of alkyl-Co bond and thus can induce radical polymerization. Moreover, alkylcobalt(III) chelates display certain advantages under conventional radical initiators. In particular, only one radical is released under decomposition of a molecule of organocobalt initiator, which diminishes the “cage” effect and enhances initiator efficiency. It is also important that organocobalt initiator allows conducting polymerizationrunrder rather mild conditions, at moderate temperatures and in aprotic media. Organocobalt complexes with CₙH₂ₙ ligands were successfully applied for polymerization of different monomers. (see. I. Ya. Levitin, A. L. Sigan, M. V. Tsikalova, M. E. Vol'pin, M. S. Tsar'kova, A. A. Kuznetsov, I. A. Gritskova, Rus. Pat. 2,070,202, Dec. 10, 1996; E. V. Rogova, M. S. Tsar'kova, I. A. Gritskova, N. P. Bessonova. Yu. K. Godovsky, Polymer Sci. Ser B, 1996; 38, 290-292; M. S. Tsar'kova, D. A. Kushlyanskii, V. A. Kryuchkov, I. A. Gritskova, Polymer Sci. Ser B, 1999, 41, 265-268; and E. I. Pisarenko, M. S. Tsar'kova, I. A. Gritskova, I. Ya. Levitin, A. L. Sigan, Polymer Sci. Ser A., 2004, 46, 16-20).

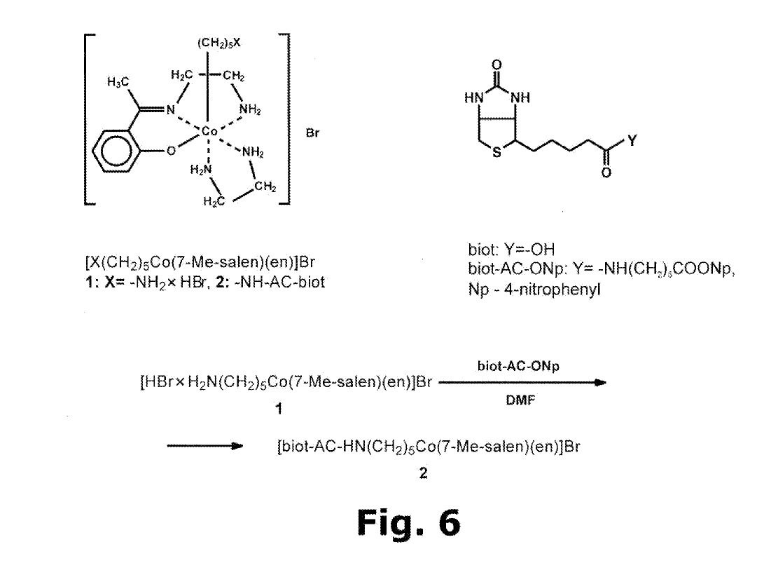

Recently, a convenient route to alkylcobalt (III) chelates containing —Br, —OH or —NH₂ at the terminal position of alkyl ligand was suggested. (see I. Ya. Levitin, A. L. Sigan, N. N. Sazikova, E. I. Pisarenko, M. S. Tsar'kova, O. A. Chumak, I. A. Gritskova, Appl. for Rus. Pat. 118650; Jun. 24, 2003). These groups may be utilized for coupling to a functional entity, which is possible later to incorporate into a polymer. Below biotinylation of the chelate with ε-aminoalkyl ligand is briefly considered (FIG. 6).

For biotiziiylation of complex [HBr×NH₂(CH₂)₅Co(7-Mesalen)(en)]Br (FIG. 6, Ex. 1), one embodiment uses a biotin derivative with 4-nitrophenyl activated carboxy group (Biot-AC-ONp), which acylates the free F-aminogroup of alkyl ligand not interacting with the other aminogroups coordinated with cobalt atom, i.e., not leading to the complex decomposition. The reaction was conducted in DMF at equimolar reagents ratio affording quantitative yield of the biotinylated product, [biot-AC-NH(CH₂)₅Co(7-Mesalen)(en)]Br (FIG. 6, Ex. 2). The complex obtained was stored in a desiccator at −20° C. in darkness for several months without decomposition.

Biotinylated organocobalt(III) chelate (see FIG. 6. Ex. 2) was used as initiator of 4-nitrophenylacrylate polymerization carried out in DMSO at 40° C. Hornolytic splitting of the Co—C bond (see FIG. 6, Ex. 1), leads to biotin-AC—NH(CH₂)₅·radical, which generate polymer chain and provide incorporation of biotin as end group into the PNPA molecules. The obtained bioti-PNFA was purified from low-molecular weight organic compounds by washing with diethyl ether, and used for further modifications.

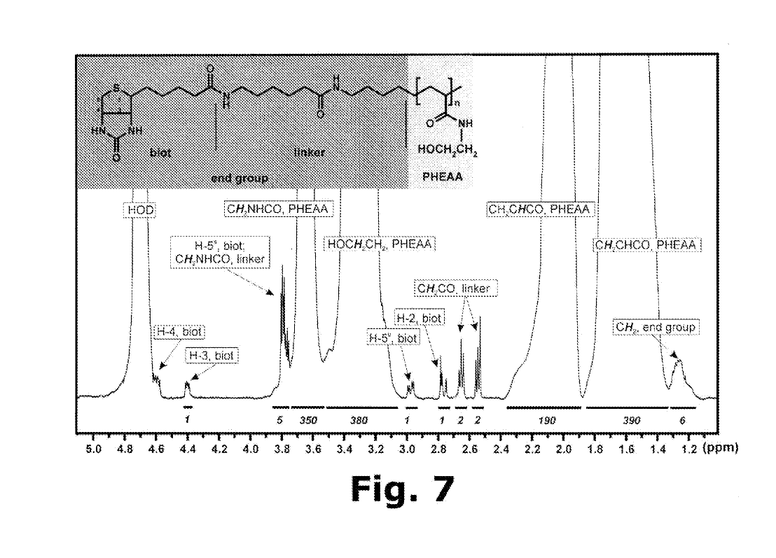

The obtained polymer was converted into a water soluble poly[(N-(2-hydroxyethyl)acrylamide], biot₁-PHEAA. The NMR spectrum of biot₁-PHEAA contained signals attributed to the biotinylated initiator fragment, and thus confirmed incorporation of biotin residues into the polymer (FIG. 7). Direct integration of the spectrum gave approximately 190:1 ratio monomer units/biotinylated end group (M/biot). However, it was found that broadening of polymer backbone peaks may lead to overestimation of their intensity, and the measured amount of biotin in the sample in reference to a low-molecular standard, 3,4-diaminobenzoic acid. The biotin content in the polymer was determined to be 52 nmol mg⁻¹, which corresponds to M/biot=160:1 (for explanation, see Example below).

In another embodiment, biot₁-PNPA also was converted into a poly(acrylic acid), whose number-average molecular weight (Mₙ) and average degree of polymerization (DP) were determined by gel-permeation chromatography (GPC). The values obtained were the following: Mₙ=15.9 kDa, DP˜220. Thus, the polymer length (DP) exceeds about 1.3 times the formal monomer-to-end group ratio (M/biot). This indicates that a part of biot₁-PNPA molecules was obtained in the result of recombination of the propagating radicals. Nonetheless, as the difference between DP and M/biot ratio is not large, it was concluded that a fraction of macromolecules with two end biotins in the synthesized polymer batch is low.

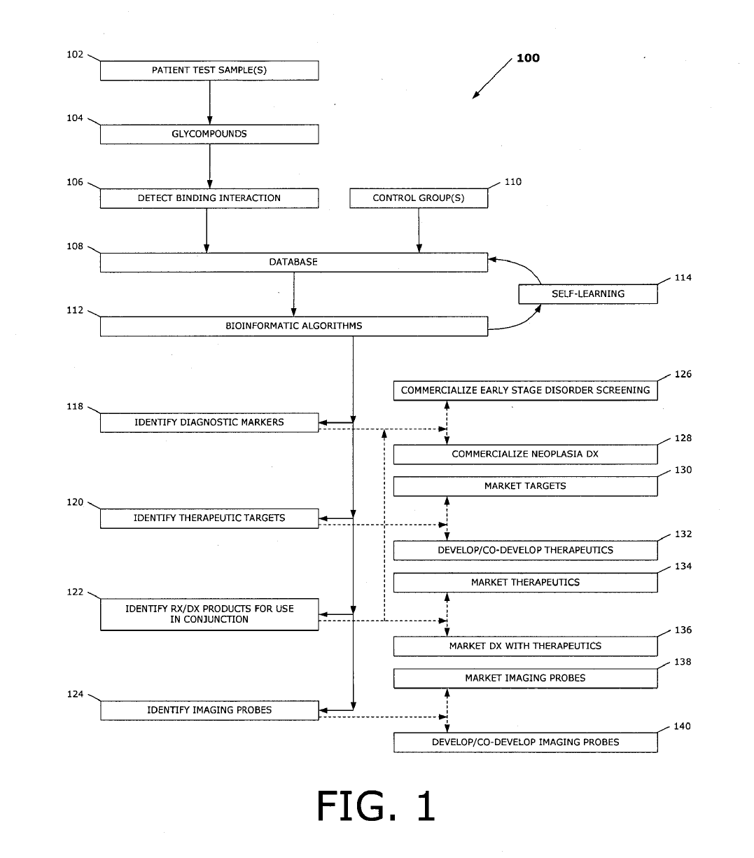

Biot₁-PNPA was used to prepare a polyacrylamide conjugate with blood group trisaccharide B (Bₜᵣᵢ), Galα1-3(Fucα1-2)Gal-(FIG. 1b, Glyc=Bₜᵣᵢ). The obtained end-labeled glycopolymer; biotₜ-PHEAA-(Bₜᵣᵢ)ₓ (molar fraction of Bₜᵣᵢ is 20%), was tested on ability to bind monoclonal antibodies (mAbs) against Bₜᵣᵢ in ELISA. Its antibody binding activity was compared to that of other biotinylated derivatives of Bₜᵣᵢ, two monomeric glycosides with Bₜᵣᵢ-biot linkers of different length and a glycopolymer, PHEAA-(Bₜᵣᵢ)ₓ-biot_{y}. The latter was synthesized by the routine approach (see FIG. 1a) and contained biotin residues randomly pendant to the polymer backbone as side groups.

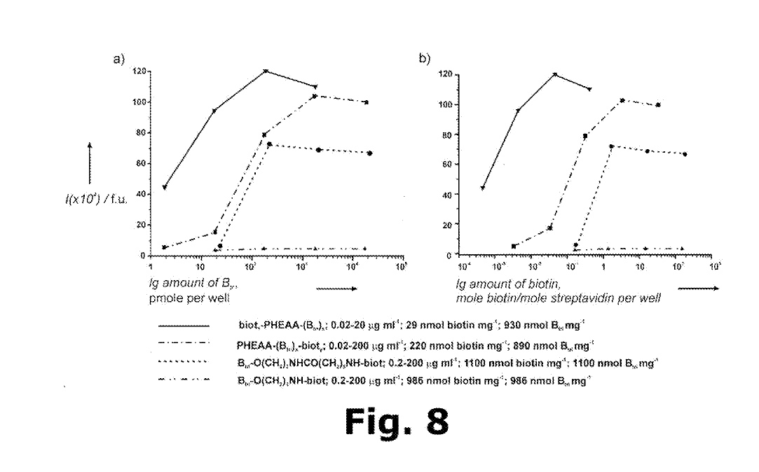

Dependence of signal in ELISA from loading of Streptaviditn-coated plates with biotynylated glycoconjugates is shown in FIG. 8. As shown, binding of antibodies with Streptavidin-coated plates modified with biotinilated glycoconjugates. The Streptavidin-coated plates were consequentially incubated with the glycoconjugates and mouse mAb; for development of the bound mAbs an anti-mouse alkaline phosphatase conjugate in combination with a fluorescent substrate were used. The detected fluorescence intensity is plotted against added per well amounts of a) Bₜᵣᵢ and b) biotin related to the quantity of Streptavidin in well. According the manufacturer 125 pmole Streptavidin is contained per well, the working well surface was calculated to be 9·10¹⁵ Å². Thus, biot₁-PHEAA-(Bₜᵣᵢ)ₓ displayed noticeable antibody binding activity when only 2 ng (4.6·10⁻⁴ mole biotin/mole Streptavidin, 1.9 pmole Bₜᵣᵢ/well) of the glycopolymer was added per well. When the same amount of PHEAA-(Bₜᵣᵢ)ₓ-biot_{y} (2 ng/well, 3.52·10⁻³ mole biotin/mole Streptavidin, 1.8 pmole Bₜᵣᵢ/well) was used for surface modification, a low signal was detected. The maximal binding of mAb with immobilized biot₁-PHEAA-(Bₜᵣᵢ)ₓ and PHEAA-(Bₜᵣᵢ)ₓ-biot_{y} was achieved at 200 ng/well (4.6·10⁻² mole biotin/mole Streptavidin, 186 pmol of Bₜᵣᵢ) and 2 μg/well (3.52 mole biotin/mole Streptavidin, 1780 pmol of Bₜᵣᵢ) loadings correspondingly, the end-labeled glycopolymer showed higher binding capacity (I˜120×10⁴ vis. 100×10⁴ f.u.). The antibody binding to surface modified with the biotinylated monovalent Bₜᵣᵢ-glycosides was noticeably lower then in the cases with the multivalent ligands. For the glycoside with a long linker, Bₜᵣᵢ-O(CH₂)₃NHCO(CH₂)₅NH-biot, assay signal was detected only when coating concentration of the glycoside exceeded 20 ng/well (0.18 mole biotin/mole Streptavidin, and 22 pmole of Bₜᵣᵢ/well). The maximal activity (I˜70×10⁴) was achieved at 220 ng/well (1.76 mole biotin/mole Streptavidin, 220 pmole of Bₜᵣᵢ/well) loading. For the glycoside with a short linker, Bₜᵣᵢ-O(CH₂)₃NH-biot, interaction with mAb was exceptionally weak (I˜5×10⁴).

To explain the result obtained, a comparison was made of the composition of the assayed glycopolymers. PNPA used for PHEAA-(Bₜᵣᵢ)ₓ-biot, preparation was converted into poly(acrylic acid), whose Mₙ=8.7 kDa (DP˜120) as estimated by GPC. Using the DP values of PNPA and biot₁-PNPA (DP˜220), a calculated number of ligands pendant to the polymer backbone was made for the corresponding glycoconjugates. Thus, the average number of Bₜᵣᵢ residues per polymer molecule (x) equals 24 for PHEAA-(Bₜᵣᵢ)ₓ-biot_{y} and 44 for biot₁-PHEAA-(Bₜᵣᵢ)ₓ. Previous experience suggests that the difference in antibody binding activity of the glycopolymers observed in ELISA cannot be caused by the minor disparity in their molecular weights and valences. Rather, it is connected with anchoring and deposition of glycopolymer molecules on Streptavidin-covered surface.

In this context it is noteworthy, that PHEAA-(Bₜᵣᵢ)₂₄-biot_{y} contains in average 6 biotin residues per molecule, any of which can interact with Streptavidin on plates surface. As already mentioned above, multipoint binding of a glycopolymer to a surface diminishes its interfacial mobility disturbing multivalent interaction with antibodies. This effect was especially noticeable when relatively small amount of the glycopolymer (loading<20 ng/well) was used to modify plate surfaces. In this case, PHEAA-(Bₜᵣᵢ)₂₄-biot₆ molecules appear to be pinned to the surface by several biotin-Streptavidin bonds. When a larger amount of PHEAA-(Bₜᵣᵢ)₂₄-biot₆ is added to the well, molecules of the glycopolyiner in average form fewer of bonds with Streptavidin, which increases the length of flexible bands within. the polymer chain and enables pendant Bₜᵣᵢ residues to adopt optimal mutual arrangement for binding with antibodies. In the case of biot₁-PHEAA-(Bₜᵣᵢ)₄₄, the problem of the restriction of the polymer chain's mobility is avoided. Even for that portion of the molecules, which contain biotin tags on the both ends; the long segment of polymer between the biotins bound to surface remains flexible. The following observations have been made: for Bₜᵣᵢ-O(CH₂)₃NHCO(CH₂)₅NH-biot₁ binding with mAb was reached maximal level when surface density of Bₜᵣᵢ was about 15 residues/1000 Å², which was close to the analogues value for biot₁-PHEAA-(Bₜᵣᵢ)₄₄ (12 residues/1000 Å²). At the same time for PHEAA-(Bₜᵣᵢ)₂₄-biot₆ the maximal binding with mAb was observed at approximately 10 times higher surface density of Bₜᵣᵢ, (˜110 residues/1000 Å²), when the negative effect of diminishing polymer chain flexibility was overwhelmed.

Thus, it has been found that a glycopolymer end-labeled with biotin is more effective for use in a glycoarrays and bioanalytical systems. The amount of biot₁-PHEAA-(Bₜᵣᵢ)₄₄ required to work at optimal sensitivity was in the range of 20-200 ng per well (˜20-200 pmole of Bₜᵣᵢ per well). Moreover, the binding capacity of end-labeled glycopolymers can be improved. It has been found that that an increase of Mₙ of a polyacrylamide scaffold from 30 to 2000 κDa leads to growth of binding activity for corresponding glycopolymer on 2-4 orders of magnitudes. Experiments showed that Mₙ of biot₁-PNPA can be easily regulated by variation of the organocobalt initiator and the monomer of concentrations in the polymerizing mixture. This finding opens the prospect for synthesis of very high valency glycoconjugates end-labeled with biotin. Another advantage of the described approach is that it allows synthesis a large batch of biot₁-PNPA (tens of grams) at one time. The latter is especially important from the bioanalytical point of view as from the same polymer-precursor with established Mₙ (DP) and molecular-mass distribution can be prepared a large number of glycopolymers deferred in nature of attached Glyc residues. Obviously, ROMP of glycosylated monomers, that according to literature could also been used for synthesis of end-labeled glycopolymers, does not allow a series of multivalent glycoconjugates with the same molecular-mass properties of a Glyc scaffold. (see R. M. Owen, J. E. Getstwicki, T. Young, L. L. Kiessling, Organic Lett., 2002, 14, 2293-2296; and E. J. Gordon, J. E. Getstwicki, L. Strong, L. L. Kiessling, Chem&Biol, 2000, 7, 9-16).

Above is describe a practical approach to synthesis of glycopolymers with end biotin groups, which are slated for introduction into the polymer scaffold during its preparation with a fragment of suitably functionalized alkylcobalt(III) chelate, the initiator with a combination of properties has not been observed for conventionlal radical initiators. The glycopolymer with biotin end group was synthesized and its high antibody binding efficacy in ELISA was demonstrated. The described polymer may be useful to construct glycoarrays and complex bio-analytical systems such as glycosylated polymer beads, liposomes and cells and the like with an engineered surface.

The glycopolymers corresponding the invention encompass macromolecules that include at least one of the carbohydrates listed in Table 1 below, or a plurality of the carbohydrates listed in Table 1.

TABLE 1
Galα
Glcα
Manα
GalNAcα
Fucα
Fucα
Rhaα
Neu5Acα
Neu5Acα
Neu5Acβ
Galβ
Glcβ
Manβ
GalNAcβ
GlcNAcβ
GlcNAcβ
GlcNGcβ
Galβ1-4GlcNAcb1-3(Galβ1-4GlcNAcb1-6)GalNAcα
GlcNAcb1-3(GlcNAcb1-6)GlcNAcb1-4GlcNAcβ
Gal[3OSO3,6OSO3]b1-4GlcNAc[6OSO3]β
Gal[3OSO3,6OSO3]b1-4GlcNAcβ
Gal[3OSO3]b1-4Glcβ
Gal[3OSO3]b1-4Glc[6OSO3]β
Gal[3OSO3]b1-4Glc[6OSO3]β
Gal[3OSO3]b1-3(Fuca1-4)GlcNAcβ
Gal[3OSO3]b1-3GalNAcα
Gal[3OSO3]b1-3GlcNAcβ
Gal[3OSO3]b1-4(Fuca1-3)GlcNAcβ
Gal[6OSO3]b1-4GlcNAcb[6OSO3]β
Gal[3OSO3]b1-4GlcNAcβ
Gal[3OSO3]b1-4GlcNAcβ
Gal[3OSO3]β
Gal[4OSO3,6OSO3]b1-4GlcNAc
Gal[4OSO3]b1-4GlcNAcβ
Man[6-H2PO3]α
Gal[6OSO3]b1-4Glcβ
Gal[6OSO3]b1-4Glcβ
Gal[6OSO3]b1-4GlcNAcβ
Gal[6OSO3]b1-4Glc[6OSO3]β
Neu5Aca2-3Gal[6OSO3]b1-4GlcNAcβ
GlcNAc[6OSO3]β
Neu5Ac[9Ac]α
Neu5Ac[9Ac]a2-6Galβ1-4GlcNAcβ
Mana1-3(Mana1-6)Manb1-4GlcNAcb1-4GlcNAcβ
GlcNAcb1-2Mana1-3(GlcNAcb1-2Mana1-6)Manb1-4GlcNAcb1-4GlcNAcβ
Galβ1-4GlcNAcb1-2Mana1-3(Galβ1-4GlcNAcb1-2Mana1-6)Manb1-4GlcNAcb1-4GlcNAcβ
Neu5Aca2-3Galβ1-4GlcNAcb1-2Mana1-3(Neu5Aca2-3Galβ1-4GlcNAcb1-2Mana1-6)Manb1-4GlcNAcb1-4GlcNAcβ
Neu5Aca2-6Galβ1-4GlcNAcb1-2Mana1-3(Neu5Aca2-6Galβ1-4GlcNAcb1-2Mana1-6)Manb1-4GlcNAcb1-4GlcNAcβ
Fucα1-2Galβ1-3GalNAcb1-3Galα
Fucα1-2Galβ1-3GalNAcb1-3Gala1-4Galβ1-4Glcβ
Fucα1-2Galβ1-3(Fucα1-4)GlcNAcβ
Fucα1-2Galβ1-3GalNAcα
Fucα1-2Galβ1-3GalNAcb1-4(Neu5Aca2-3)Galβ1-4Glcβ
Fucα1-2Galβ1-3GalNAcb1-4(Neu5Aca2-3)Galβ1-4Glcβ
Fucα1-2Galβ1-3GlcNAcb1-3Galβ1-4Glcβ
Fucα1-2Galβ1-3GlcNAcb1-3Galβ1-4Glcβ
Fucα1-2Galβ1-3GlcNAcβ
Fucα1-2Galβ1-3GlcNAcβ
Fucα1-2Galβ1-4(Fucα1-3)GlcNAcb1-3Galβ1-4(Fucα1-3)GlcNAcβ
Fucα1-2Galβ1-4(Fucα1-3)GlcNAcb1-3Galβ1-4(Fucα1-3)GlcNAcb1-3Galβ1-4(Fucα1-3)GlcNAcβ
Fucα1-2Galβ1-4(Fucα1-3)GlcNAcβ
Fucα1-2Galβ1-4(Fucα1-3)GlcNAcβ
Fucα1-2Galβ1-4GlcNAcb1-3Galβ1-4GlcNAcβ
Fucα1-2Galβ1-4GlcNAcb1-3Galβ1-4GlcNAcb1-3Galβ1-4GlcNAcβ
Fucα1-2Galβ1-4GlcNAcβ
Fucα1-2Galβ1-4GlcNAcβ
Fucα1-2Galβ1-4Glcβ
Fucα1-2Galβ
Fucα1-2GlcNAcβ
Fucα1-3GlcNAcβ
Fucα1-4GlcNAcβ
Fucα1-3GlcNAcβ
GalNAca1-3(Fucα1-2)Galβ1-3GlcNAcβ
GalNAca1-3(Fucα1-2)Galβ1-4(Fucα1-3)GlcNAcβ
GalNAca1-3(Fucα1-2)Galβ1-4GlcNAcβ
GalNAca1-3(Fucα1-2)Galβ1-4GlcNAcβ
GalNAca1-3(Fucα1-2)Galβ1-4Glcβ
GalNAca1-3(Fucα1-2)Galβ
GalNAca1-3GalNAcβ
GalNAca1-3Galβ
GalNAca1-4(Fucα1-2)Galβ1-4GlcNAcβ
GalNAcb1-3GalNAcα
GalNAcb1-3(Fucα1-2)Galβ
GalNAcb1-3Gala1-4Galβ1-4GlcNAcβ
GalNAcb1-4(Fucα1-3)GlcNAcβ
GalNAcb1-4GlcNAcβ
GalNAcb1-4GlcNAcβ
Gala1-2Galβ
Gala1-3(Fucα1-2)Galβ1-3GlcNAcβ
Gala1-3(Fucα1-2)Galβ1-4(Fucα1-3)GlcNAcβ
Gala1-3(Fucα1-2)Galβ1-4GlcNAcβ
Gala1-3(Fucα1-2)Galβ1-4Glcβ
Gala1-3(Fucα1-2)Galβ
Gala1-3(Gala1-4)Galβ1-4GlcNAcβ
Gala1-3GalNAcα
Gala1-3GalNAcβ
Gala1-3Galβ1-4(Fucα1-3)GlcNAcβ
Gala1-3Galβ1-3GlcNAcβ
Gala1-3Galβ1-4GlcNAcβ
Gala1-3Galβ1-4Glcβ
Gala1-3Galβ
Gala1-4(Fucα1-2)Galβ1-4GlcNAcβ
Gala1-4Galβ1-4GlcNAcβ
Gala1-4Galβ1-4GlcNAcβ
Gala1-4Galβ1-4Glcβ
Gala1-4GlcNAcβ
Gala1-6Glcβ
Galβ1-2Galβ
Galβ1-3(Fucα1-4)GlcNAcb1-3Galβ1-4(Fucα1-3)GlcNAcβ
Galβ1-3(Fucα1-4)GlcNAcb1-3Galβ1-4GlcNAcβ
Galβ1-3(Fucα1-4)GlcNAcβ
Galβ1-3(Fucα1-4)GlcNAcβ
Galβ1-3(Fucα1-4)GlcNAcβ
Galβ1-3(Galβ1-4GlcNAcb1-6)GalNAcα
Galβ1-3(GlcNAcb1-6)GalNAcα
Galβ1-3(Neu5Aca2-6)GalNAcα
Galβ1-3(Neu5Acb2-6)GalNAcα
Galβ1-3(Neu5Aca2-6)GlcNAcb1-4Galβ1-4Glcβ
Galβ1-3GalNAcα
Galβ1-3GalNAcβ
Galβ1-3GalNAcb1-3Gala1-4Galβ1-4Glcβ
Galβ1-3GalNAcb1-4(Neu5Aca2-3)Galβ1-4Glcβ
Galβ1-3GalNAcb1-4Galβ1-4Glcβ
Galβ1-3Galβ
Galβ1-3GlcNAcb1-3Galβ1-4GlcNAcβ
Galβ1-3GlcNAcb1-3Galβ1-4Glcβ
Galβ1-3GlcNAcβ
Galβ1-3GlcNAcβ
Galβ1-4(Fucα1-3)GlcNAcβ
Galβ1-4(Fucα1-3)GlcNAcβ
{Galβ1-4(Fucα1-3)GlcNAcb1-3}2
{Galβ1-4(Fucα1-3)GlcNAcb1-3}3
Galβ1-4Glc[6OSO3]β
Galβ1-4Glc[6OSO3]β
Galβ1-4GalNAca1-3(Fucα1-2)Galβ1-4GlcNAcβ
Galβ1-4GalNAcb1-3(Fucα1-2)Galβ1-4GlcNAcβ
Galβ1-4GlcNAcb1-3(Galβ1-4GlcNAcb1-6)GalNAcα
Galβ1-4GlcNAcb1-3GalNAcα
Galβ1-4GlcNAcb1-3Galβ1-4(Fucα1-3)GlcNAcb1-3Galβ1-4(Fucα1-3)GlcNAcβ
{Galβ1-4GlcNAcb1-3}3
Galβ1-4GlcNAcb1-3Galβ1-4GlcNAcβ
Galβ1-4GlcNAcb1-3Galβ1-4Glcβ
Galβ1-4GlcNAcb1-3Galβ1-4Glcβ
Galβ1-4GlcNAcb1-6(Galβ1-3)GalNAcα
Galβ1-4GlcNAcb1-6GalNAcα
Galβ1-4GlcNAcβ
Galβ1-4GlcNAcβ
Galβ1-4Glcβ
Galβ1-4Glcβ
GlcNAcb1-3Galβ1-4GlcNAcβ
GlcNAca1-6Galβ1-4GlcNAcβ
GlcNAcb1-2Galβ1-3GalNAcα
GlcNAcb1-3(GlcNAcb1-6)GalNAcα
GlcNAcb1-3(GlcNAcb1-6)Galβ1-4GlcNAcβ
GlcNAcb1-3GalNAcα
GlcNAcb1-3Galβ
GlcNAcb1-3Galβ1-3GalNAcα
GlcNAcb1-3Galβ1-4GlcNAcβ
GlcNAcb1-3Galβ1-4GlcNAcβ
GlcNAcb1-3Galβ1-4GlcNAcb1-3Galβ1-4GlcNAcβ
GlcNAcb1-3Galβ1-4Glcβ
GlcNAcb1-4MDPLys (bact.cell wall)
GlcNAcb1-4(GlcNAcb1-6)GalNAcα
GlcNAcb1-4Galβ1-4GlcNAcβ
GlcNAcb1-4GlcNAcb1-4GlcNAcb1-4GlcNAcb1-4GlcNAcb1-4GlcNAcβ
GlcNAcb1-4GlcNAcb1-4GlcNAcb1-4GlcNAcb1-4GlcNAcβ
GlcNAcb1-4GlcNAcb1-4GlcNAcβ
GlcNAcb1-6(Galβ1-3)GalNAcα
GlcNAcb1-6GalNAcα
GlcNAcb1-6Galβ1-4GlcNAcβ
Glca1-4Glcβ
Glca1-4Glcα
Glca1-6Glca1-6Glcβ
Glcb1-4Glcβ
Glcb1-6Glcβ
HOCH2(HOCH)4CH2NH2; G-ol-amine:
GlcAα
GlcAβ
GlcAb1-3Galβ
GlcAb1-6Galβ
KDNa2-3Galβ1-3GlcNAcβ
KDNa2-3Galβ1-4GlcNAcβ
Mana1-2Mana1-2Mana1-3Manα
Mana1-2Mana1-3(Mana1-2Mana1-6)Manα
Mana1-2Mana1-3Manα
Mana1-6(Mana1-2Mana1-3)Mana1-6(Mana1-2Mana1-3)Manb1-4GlcNAcb1-4GlcNAcβ
Mana1-2Mana1-6(Mana1-3)Mana1-6(Mana1-2Mana1-2Mana1-3)Manb1-4GlcNAcb1-4GlcNAcβ
Mana1-2Mana1-2Mana1-3[Mana1-2Mana1-3(Mana1-2Mana1-6)Mana1-6]Manb1-4GlcNAcb1-4GlcNAcβ
Mana1-3(Mana1-6)Manα
Mana1-3(Mana1-2Mana1-2Mana1-6)Manα
Mana1-2Mana1-3(Mana1-3(Mana1-6)Mana1-6)Manb1-4GlcNAcb1-4GlcNAcβ
Mana1-3(Mana1-3(Mana1-6)Mana1-6)Manb1-4GlcNAcb1-4GlcNAcβ
Mixture of Man 5 to Man 9-Asn
Manb1-4GlcNAcβ
Neu5Aca2-3(Galβ1-3GalNAcb1-4)Galβ1-4Glcβ
Neu5Aca2-3Galβ1-3GalNAcα
Neu5Aca2-8Neu5Aca2-8Neu5Aca2-8Neu5Aca2-3(GalNAcb1-4)Galβ1-4Glcβ
Neu5Aca2-8Neu5Aca2-8Neu5Aca2-3(GalNAcb1-4)Galβ1-4Glcβ
Neu5Aca2-8Neu5Aca2-8Neu5Aca2-3Galβ1-4Glcβ
Neu5Aca2-8Neu5Aca2-3(GalNAcb1-4)Galβ1-4Glcβ
Neu5Aca2-8Neu5Aca2-8Neu5Acα
Neu5Aca2-3Gal[6OSO3]b1-4(Fucα1-3)GlcNAcβ
Neu5Aca2-3(GalNAcb1-4)Galβ1-4GlcNAcβ
Neu5Aca2-3(GalNAcb1-4)Galβ1-4GlcNAcβ
Neu5Aca2-3(GalNAcb1-4)Galβ1-4Glcβ
Neu5Aca2-3(Neu5Aca2-3Galβ1-3GalNAcb1-4)Galβ1-4Glcβ
Neu5Aca2-3(Neu5Aca2-6)GalNAcα
Neu5Aca2-3GalNAcα
Neu5Aca2-3GalNAcb1-4GlcNAcβ
Neu5Aca2-3Gal[6OSO3]b1-3GlcNAc
Neu5Aca2-3Galβ1-3(Fucα1-4)GlcNAcβ
Neu5Aca2-3Galβ1-3(Fucα1-4)GlcNAcb1-3Galβ1-4(Fucα1-3)GlcNAcβ
Neu5Aca2-3Galβ1-3(Neu5Aca2-3Galβ1-4)GlcNAcβ
Neu5Aca2-3Galβ1-3GalNAc[6OSO3]α
Neu5Aca2-3(Neu5Aca2-6)GalNAcα
Neu5Aca2-3Galβ
Neu5Aca2-3Galβ1-3GalNAcb1-3Galβ1-4Galβ1-4Glcβ
Neu5Aca2-3Galβ1-3GalNAcb1-3Galβ1-4GlcNAcβ
Neu5Aca2-3Galβ1-3GlcNAcβ
Neu5Aca2-3Galβ1-3GlcNAcβ
Neu5Aca2-3Galβ1-4GlcNAc[6OSO3]β
Neu5Aca2-3Galβ1-4(Fucα1-3)GlcNAc[6OSO3]β
Neu5Aca2-3Galβ1-4(Fucα1-3)GlcNAcb1-3Galβ1-4(Fucα1-3)GlcNAcb1-3Galβ1-4(Fucα1-3)GlcNAcβ
Neu5Aca2-3Galβ1-4(Fucα1-3)GlcNAcβ
Neu5Aca2-3Galβ1-4(Fucα1-3)GlcNAcβ
Neu5Aca2-3Galβ1-4(Fucα1-3)GlcNAcb1-3Galβ
Neu5Aca2-3Galβ1-4(Fucα1-3)GlcNAcb1-3Galβ1-4GlcNAcβ
Neu5Aca2-3Galβ1-4GlcNAcb1-3Galβ1-4(Fucα1-3)GlcNAc
Neu5Aca2-3Galβ1-4GlcNAcb1-3Galβ1-4GlcNAcb1-3Galβ1-4GlcNAcβ
Neu5Aca2-3Galβ1-4GlcNAcβ
Neu5Aca2-3Galβ1-4GlcNAcβ
Neu5Aca2-3Galβ1-4GlcNAcb1-3Galβ1-4GlcNAcβ
Neu5Aca2-3Galβ1-4Glcβ
Neu5Aca2-3Galβ1-4Glcβ
Neu5Aca2-6(Galβ1-3)GalNAcα
Neu5Aca2-6GalNAcα
Neu5Aca2-6GalNAcb1-4GlcNAcβ
Neu5Aca2-6Galβ1-4GlcNAc[6OSO3]β
Neu5Aca2-6Galβ1-4GlcNAcβ
Neu5Aca2-6Galβ1-4GlcNAcβ
Neu5Aca2-6Galβ1-4GlcNAcb1-3Galβ1-4(Fucα1-3)GlcNAcb1-3Galβ1-4(Fucα1-3)GlcNAcβ
Neu5Aca2-6Galβ1-4GlcNAcb1-3Galβ1-4GlcNAcβ
Neu5Aca2-6Galβ1-4Glcβ
Neu5Aca2-6Galβ1-4Glcβ
Neu5Aca2-6Galβ
Neu5Aca2-8Neu5Acα
Neu5Aca2-8Neu5Aca2-3Galβ1-4Glcβ
Neu5Acb2-6GalNAcα
Neu5Acb2-6Galβ1-4GlcNAcβ
Neu5Acb2-6(Galβ1-3)GalNAcα
Neu5Gca2-3Galβ1-3(Fucα1-4)GlcNAcβ
Neu5Gca2-3Galβ1-3GlcNAcβ
Neu5Gca2-3Galβ1-4(Fucα1-3)GlcNAcβ
Neu5Gca2-3Galβ1-4GlcNAcβ
Neu5Gca2-3Galβ1-4Glcβ
Neu5Gca2-6GalNAcα
Neu5Gca2-6Galβ1-4GlcNAcβ
Neu5Gcα

The glycopolymerzs alternatively can comprise at least one macromolecule listed in Tables 1-3 and other tables in PCT/US2005/007370 filed Mar. 7, 2005 titled “High Throughput Glycan Microarrays”; and U.S. Provisional Patent Application No. 60/629,666 filed Nov. 19, 2004 titled “Development of Blood Based Test Allowing Diagnosis of Neoplasia Status”.

In another embodiment, any conjugate from a group identified above is provided with an additional fluorescent label which can be bound to the conjugate as is known in the art. The fluorescent label is used in methods for quantitative control of immobilization and evaluation of the substance amount in a solution.

In another embodiment, any conjugate from a group identified above included at least two different residues of oligosaccharide and/or a combination oligosaccharide/noncarbohydrate i.e., complex epitopes.

The present invention further pertains to a packaged glycopolymer provided in a kit or other container for detecting, controlling, preventing or treating a neoplasia (e.g., ovarian cancer) or other disorder. In one exemplary embodiment, the kit or container holds an array or library of glycopolymers or glycalns (e.g., a glycopolymer coupled to a single biotin molecule) for detecting ovarian cancer and instructions for using the array or library of glycans for detecting the ovarian cancer. The array includes at least one glycan that is bound by antibodies present in serum samples of an ovarian cancer patient.

In another embodiment, the kit or container holds a therapeutically effective amount of a pharmaceutical composition for controlling a neoplasm (e.g., ovarian cancer) and instructions for using the pharmaceutical composition for control of the neoplasm. The pharmaceutical composition includes at least one glycopolymer or glycan of the present invention, in a therapeutically effective amount such that the neoplasm is controlled, prevented or treated.

In a further embodiment, the kit comprises a container containing an antibody that specifically binds to a glycopolymer or glycan that is associated with neoplasia (e.g., ovarian cancer or metastatic ovarian cancer). The antibody can have a directly attached or indirectly associated therapeutic agent. The antibody can also be provided in liquid form, powder form or other form permitting ready administration to a patient.

The kits of the invention can also comprise containers with tools useful for administering the compositions of the invention. Such tools include syringes, swabs, catheters, antiseptic solutions and the like.

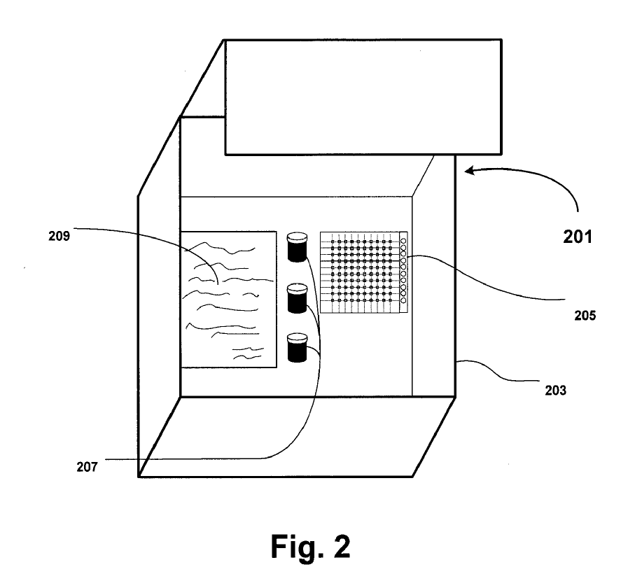

As described herein and shown in FIG. 2, in certain embodiments a kit 201 can include a housing or container 203 for housing various components. As shown in FIG. 2, and described herein, the kit 201 can optionally include libraries and/or arrays of glycopolymers 205, instructions 209 and reagents 207. Other embodiments of the kit 201 are envisioned wherein the components include various additional features described herein.

In another embodiment, a result obtained using the glycopolymers described herein is used for detection/treatment/prevention of early stage diseases and/or neoplasia of an individual, for example, a patient. In a further embodiment, the method of detection/ treatment/ prevention of early stage diseases and/or neoplasia includes reviewing or analyzing data relating to the presence of, for example, circulating antibodies that react with neoplasia-related epitopes in a sample. A conclusion is then provided to a patient, a health care provider or a health care manager, the conclusion being based on the review or analysis of data regarding a disease diagnosis or early stage disease detection. It is envisioned that in another embodiment that providing a conclusion to a patient, a health care provider or a health care manager includes transmission of the data over a network.

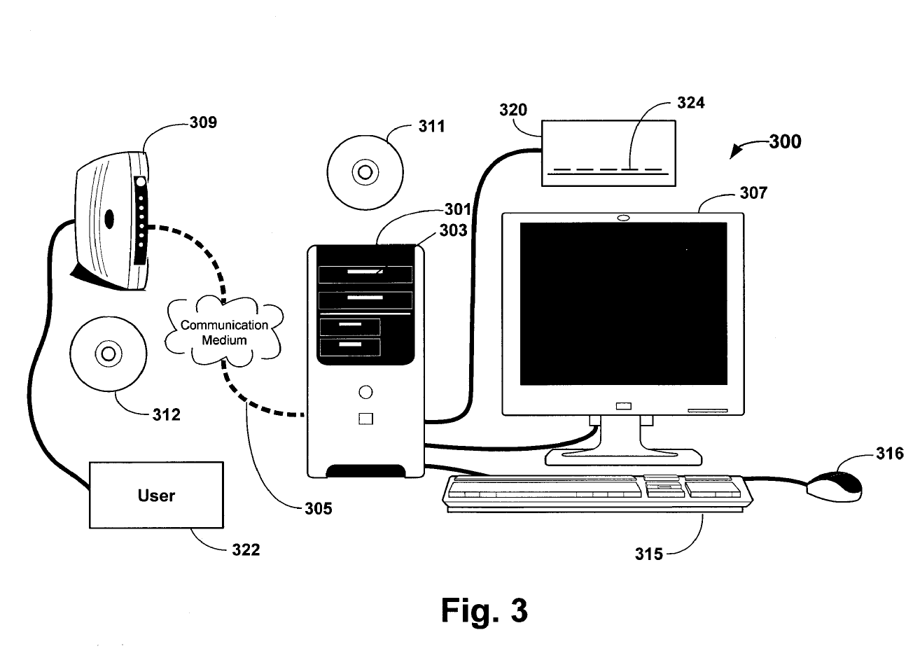

FIG. 3 is a block diagram showing a representative example logic device through which reviewing or analyzing data relating to the present invention can be achieved. Such data can be in relation to detection/ treatment/ prevention of early stage diseases and/or neoplasia in an individual. FIG. 3 shows a computer system (or digital device) 300 connected to an apparatus 320 for use with libraries and arrays of glycans 324 to, for example, produce a result. The computer system 300 may be understood as a logical apparatus that can read instructions from media 311 and/or network port 305, which can optionally be connected to server 309 having fixed-media 312. The system shown in FIG. 3 includes CPU 301, disk drives 303, optional input devices such as keyboard 315 and/or mouse 316 and optional monitor 307. Data communication can be achieved through the indicated communication medium to a server 309 at a local or a remote location. The communication medium can include any means of transmitting and/or receiving data. For example, the communication medium can be a network connection, a wireless connection or an internet connection. Such a connection provide for communication over the World Wide Web. It is envisioned that data relating to the present invention can be transmitted over such networks or connections for reception and/or review by a party 322. The receiving party 322 can be a patient, a health care provider or a health care manager.

In one embodiment, a computer-readable medium includes a medium suitable for transmission of a result of an analysis of a biological sample. The medium can include a result regarding detection/ treatment/ prevention of early stage diseases and/or neoplasia of a subject, wherein such a result is derived using the methods described herein.

The glycopolymers of the invention can be included in devices or in systems, for example a biosensing system, useful for monitoring, diagnosing and/or prognosing of a health state of a subject, for example based on a subject test sample. In one embodiment a glycopolymer of the invention is used with a flow cytometry system including a plurality of beads carrying the one or more glycopolymer, using flow cytometry to detect a binding interaction between antibodies in the subject test sample and the glycopolymer, and utilizing an algorithm to identify a pattern of binding interactions previously identified as being associated with a neoplasia (e.g. a cancer) or risk of neoplasia (e.g., cancer). It is envisioned that the glycopolymer can include a plurality of different glycopolymers.

As discussed herein, in one aspect the invention relates to, for example, diagnostic screening of risk of neoplasia, the existence of neoplasia in a patient or the monitoring of treatment associated with neoplasia. Neoplasia is generally defined as abnormal, disorganized growth in a tissue or organ. Such a growth can be in the form of a mass, often called a neoplasm, tumor or cancer. Neoplasms can be benign or malignant lesions. Malignant lesions are often called cancer. The National Institute of Health lists thirteen cancers as the most frequently diagnosed in the United States, each having an estimated annual incidence for 2006 at 30,000 cases or more. These most frequently diagnosed cancers include: bladder cancer, melanoma, breast cancer, non-Hodgkin's lymphoma, colon and rectal cancer, pancreatic cancer, endometrial cancer, prostate cancer, kidney (renal cell) cancer, skin cancer (non-melanoma), leukemia, thyroid cancer and lung cancer. Source: http://www.cancer.gov/cancertopics/commoncancers. Last accessed Sep. 12, 2006.

An extensive listing of cancer types includes but is not limited to acute lymphoblastic leukemia (adult), acute lymphoblastic leukemia (childhood), acute myeloid leukemia (adult), acute myeloid leukemia (childhood), adrenocortical carcinoma, adrenocortical carcinoma (childhood), AIDS-related cancers, AIDS-related lymphoma, anal cancer, astrocytoma (childhood cerebellar), astrocytoma (childhood cerebral), basal cell carcinoma, bile duct cancer (extrahepatic), bladder cancer, bladder cancer (childhood), bone cancer (osteosarcoma/malignant fibrous histiocytoma), brain stem glioma (childhood), brain tumor (adult), brain tumor—brain stem glioma (childhood), brain tumor—cerebellar astrocytoma (childhood), brain tumor—cerebral astrocytoma/malignant glioma (childhood), brain tumor—ependymoma (childhood), brain tumor—medulloblastoma (childhood), brain tumor—supratentorial primitive neuroectodermal tumors (childhood), brain tumor—visual pathway and hypothalamic glioma (childhood), breast cancer (female, male, childhood), bronchial adenomas/carcinoids (childhood), Burkitt's lymphoma, carcinoid tumor (childhood), carcinoid tumor (gastrointestinal), carcinoma of unknown primary site (adult and childhood), central nervous system lymphoma (primary), cerebellar astrocytoma (childhood), cerebral astrocytoma/malignant glioma (childhood), cervical cancer, chronic lymphocytic leukemia, chronic myclogenous leukemia, chronic myeloproliferative disorders, colon cancer, colorectal cancer (childhood), cutaneous t-cell lymphoma, endometrial cancer, ependymoma (childhood), esophageal cancer, esophageal cancer (childhood), Ewing's family of tumors, extracranial germ cell tumor (childhood), extragonadal germ cell tumor, extrahepatic bile duct cancer, eye cancer (intraocular melanoma and retinoblastoma), gallbladder cancer, gastric (stomach) cancer, gastric (stomach) cancer (childhood), gastrointestinal carcinoid tumor, gastrointestinal stromal tumor (gist), germ cell tumor (extracranial (childhood), extragonadal, ovarian), gestational trophoblastic tumor, glioma (adult), glioma (childhood: brain stem, cerebral astrocytoma, visual pathway and hypothalamic), hairy cell leukemia, head and neck cancer, hepatocellular (liver) cancer (adult primary and childhood primary), Hodgkin's lymphoma (adult and childhood), Hodgkin's lymphoma during pregnancy, hypopharyngeal cancer, hypothalamic and visual pathway glioma (childhood), intraocular melanoma, islet cell carcinoma (endocrine pancreas), Kaposi's sarcoma, kidney (renal cell) cancer, kidney cancer (childhood), laryngeal cancer, laryngeal cancer (childhood), leukemia—acute lymphoblastic (adult and childhood), leukemia, acute myeloid (adult and childhood), leukemia—chronic lymphocytic, leukemia—chronic myelogenous, leukemia—hairy cell, lip and oral cavity cancer, liver cancer (adult primary and childhood primary), lung cancer—non-small cell, lung cancer—small cell, lymphoma—AIDS-related, lymphoma—Burkitt's, lymphoma—cutaneous t-cell, lymphoma—Hodgkin's (adult, childhood and during pregnancy), lymphoma—non-Hodgkin's (adult, childhood and during pregnancy), lymphoma—primary central nervous system, macroglobulinemia—Waldenström's, malignant fibrous histiocytoma of bone/osteosarcoma, medulloblastoma (childhood), melanoma, melanoma—intraocular (eye), Merkel cell carcinoma, mesothelioma (adult) malignant, mesothelioma (childhood), metastatic squamous neck cancer with occult primary, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia, chronic, myeloid leukemia (adult and childhood) acute, myeloma—multiple, rhyeloproliferative disorders—chronic, nasal cavity and paranasal sinus cancer, nasopharyngeal cancer, nasopharyngeal cancer (childhood), neuroblastoma, non-small cell lung cancer, oral cancer (childhood), oral cavity and lip cancer, oropharyngeal cancer, osteosarcoma/malignant fibrous histiocytoma of bone, ovarian cancer (childhood), ovarian epithelial cancer, ovarian germ cell tumor, ovarian low malignant potential tumor, pancreatic cancer, pancreatic cancer (childhood), pancreatic cancer—islet cell, paranasal sinus and nasal cavity cancer, parathyroid cancer, penile cancer, pheochromocytoma, pineoblastoma and supratentorial primitive neuroectodermal tumors (childhood), pituitary tumor, plasma cell neoplasm/multiple myeloma, pleuropulmonary blastoma, pregnancy and breast cancer, primary central nervous system lymphoma, prostate cancer, rectal cancer, renal cell (kidney) cancer, renal cell (kidney) cancer (childhood), renal pelvis and ureter—transitional cell cancer, retinoblastoma, rhabdomyosarcoma (childhood), salivary gland cancer, salivary gland cancer (childhood), sarcoma—Ewing's family of tumors, sarcoma—Kaposi's, sarcoma—soft tissue (adult and childhood), sarcoma—uterine, Sézary syndrome, skin cancer (non-melanoma), skin cancer (childhood), skin cancer (melanoma), skin carcinoma—Merkel cell, small cell lung cancer, small intestine cancer, soft tissue sarcoma (adult and childhood), squamous cell carcinoma, squamous neck cancer with occult primary—metastatic, stomach (gastric) cancer, stomach (gastric) cancer (childhood), supratentorial primitive neuroectodermal tumors (childhood), testicular cancer, thymoma (childhood), thymonia and thymic carcinoma, thyroid cancer, thyroid cancer (childhood), transitional cell cancer of the renal pelvis and ureter, trophoblastic tumor, gestational, ureter and renal pelvis -transitional cell cancer, urethral cancer, uterine cancer—endometrial, uterine sarcoma, vaginal cancer, visual pathway and hypothalamic glioma (childhood), vulvar cancer, Waldenström's macroglobulinemia, and Wilms' tumor. Source: http://www.cancer.gov/cancertopics/alphalist. Last accessed Sep. 12, 2006.

Accordingly, the present glycopolymers and methods of use thereof are very useful because various embodiments of the invention can be used in conjunction with monitoring, diagnosing and/or prognosing a host of neoplasms. In addition to monitoring, diagnosing and/or prognosing neoplasia, the glycopolymers and methods of use are valuable and useful in treatment/prevention of early stage diseases and/or neoplasia as discussed in U.S. provisional patent application No. 60/871,381, filed Dec. 21, 2006, titled Bioanalytical System Business Methods.

The business of diagnostics and therapeutics research and development includes the discovery of biomarkers and targets through to final product launch. The development processes are very lengthy, expensive and involve a high risk. On average, it takes over a decade to develop a therapeutic drug product from the initial research stage to FDA approval. The cost of developing and commercializing a potential drug can cost $500 million to $1 billion or more. Any new business methods that can accelerate the development cycle of a potential drug, accelerate commercialization or reduce risk can bring significant financial benefits to the affected company that develops the new methods. Therefore, business systems and methods that improve the efficiency and timeliness of regulatory approval are highly valuable.

The business systems and methods herein include, for example, the development of bioanalytic systems based on carbohydrate sensing of binding interactions with patient test samples, which can be any body fluid or even respiration. FIG. 1 is a block diagram that illustrates exemplary steps in business systems and methods herein.

The bioanalytic systems corresponding to the invention provide capabilities for identifying commercially valuable biomarkers and therapeutic targets, and verifying such results using associations studies. The biomarker and targets can be marketed to acquire up-front fees, co-development and research payments, milestone payments, database subscriptions, product sales, royalties and the like, all of which can contribute revenue to the business model. Data obtained via the bioanalytic method can further be used, for example, for association studies and can further be licensed to biotechnology, pharmaceutical, or other interested parties on an exclusive field of use or non-exclusive basis. In addition or alternatively, revenue can be generated by entering into discovery contracts on an exclusive or non-exclusive basis with biotech, pharmaceutical, or other companies that are interested in pharmacoglycomic fields used to verify existing drug target candidates, to monitor drug response in trials, to screen candidates for trials and the like.

### EXAMPLES

All chemicals and solvents apart from those mentioned below were purchased from Merck (Germany) and Fluka (Switzerland). The solvents were dried and purified by conventional procedures. Galα1-3(Fucα1-2)Galβ-O(CH₂)₃NH₂, 4-Nitrophenylacrylate, PNPA and PHEAA-(Bₜᵣᵢ)ₓ-biotin_{y} were synthesized as described elsewhere. Mouse monoclonal antibodies (mAb) B8 against Bₜᵣᵢ were obtained from All-Russian Hematology Research Center, Moscow. Anti-mouse IgG+IgM (H+L)-alkaline phosphatase conjugate (Ig-AP) was the product of AP Biotech (UK). The organocobalt complexes used in this work were protected from light; their solutions were concentrated under vacuum with bath temperature kept below 35° C.

TLC was performed on silica gel covered plates “Kieselgel 60” (E. Merck, Germany). Capillary electrophoresis (CE) analysis was performed on the CE System BioFocus 3000 (Bio-Rad, USA) using a fused silica capillary 17 cm×0.25 μm (i.d.) with internal linear polyacrylamide cladding; capillary and sample carousel were thermostated at 20° C. and 7° C. respectively; detection wavelength: 310 nm; buffer: a 4:1 (v/v) mixture of methanol with 0.025 M aqueous acetic acid adjusted to pH 7.25 with ethylenediamine. The applied voltage used was 14 kV.

Analytical gel-permeating chromatography (GPC) was carried by HPLC using a TSK-4000SW column, 7.5×300 mm, (Ultrapack, Sweden); mobile phase: 0.2 M NaCl, flow rate −1 mL/min; UV detection at 210 nm. The column was calibrated with a set of PAA calibration kit, Mₙ=1.25−1.100 κDA (Polymer Lab., USA).

Proton nuclear magnetic resonance (¹H NMR) spectra were recorded on Bruker WM 500, T=303 K, solvent-CD₃OD (δ=4.500), D₂O (4.750) and CDCl₃ (δ=7.270) were used as solvents. Signal assignment for the complex 2 was performed using ¹H-¹H COSY technique.

[biot-NH(CH₂)₅Co(7-Me-salen)(en)]Br(2):

[HBr×NH2(CH2)5Co(7-Mesalen)(en)]Br (100 mg, 216 μmol) prepared as described in [12] was dissolved in DMF (5 mL), to the solution were added Biot-AC-ONp (79 mg, 216 μmol) and NEt3 (60 μL, 432 μmol). Reaction mixture was protected from light and kept 24 h at room temperature. Product was purified by size-exclusion chromatography, Sephadex LH-20, elution—0.05 M NH₃ in MeOH, combined fractions containing product were evaporated and the remains was dried in vacuum. Yield—116 mg (87%); a red-brown solid; TLC: R_{f}0.7,.eluent—EtOH/Py/H₂O/AcOH 3:1:1:1; ¹H NMR (CD₃OD, 500 MHz): 7.67 (dd, 1H, ^{H-3}J=8.6 Hz, ^{H-4}J=_{1.7} Hz, H-5Ar), 7.17 (ddd, 1H, ^{H-4}J=13.7 Hz, ^{H-2}J=1.1 Hz, H-3 Ar), 6.84 (dd, 1H, ^{H-3}J=1.5 Hz, ^{H-2} Ar), 6.63 (dd, 1H, H-4 Ar), 4.68 (ddd, 1H, ^{H-3}J=7.9 Hz, ^{H-5a}J=5.0 Hz, ^{H-5b}J=1.0 Hz, H-4 biot), 4.49 (dd, ^{H-2}J=4.5 Hz, H-3 biot), 3.84 (ddd, 1H, ^{NHb}J=14.4 Hz, ^{CHa}J=5.7 Hz, ^{CHb}J=4.0 Hz, NH^{a}CH₂CH₂N═), 3.69-3.60 (m, 1H, NH^{b}CH₂CH₂N═), 3.54-3.46 in CD₃OH (˜2H, NH₂CH₂ EDA), 3.37-3.32 (br. m, 8H, NH₂CH₂ EDA, 2×CH₂NH, CH₂Co), 3.17 (ddd, 1H, ^{CHb}J=18.6 Hz, J=11 Hz, J=3.8 Hz, CH₂CH^{a}N═), 3.12 (dd, 1H, ^{H-5b}J=12.6 Hz, H-5a biot), 3.04-2.86 (br m, 4H, CH₂CH^{b}N═, CH₂NH₂EDA, H-5b biot), 2.80-2.71 (m, 1H, CHNH₂ EDA), 2.71-2.59 (m, 5H, N═CCH₃, NH₂CH^{a}CH₂N═, CHNH₂, EDA), 2.51-2.41 (m, NH₂CH₂CH^{b}N═), 2.40-2.31 (m, 2×2H, CH₂CO), 1.98-1.75 9 (br m, 6H, 3×CH₂), 1.74-1.59 (br m, 8H, 4×CH₂), 1.57-1.39 (br m, 4H, 2×CH₂); CE: single peak with a migration time 11.5 min was detected.

Biot₁-PNPA:

A solution of 4-nitrophenylacrylate (200 mg, 1.03 mmol) and 2 (2 mg, 2.49 μmol) in DMSO (1 mL) was placed into a glass vial, which was sealed under vacuum after degassing of the contents via three freeze-pump-thaw cycles. The reaction mixture was kept for 24 h under 40° C., the vial was opened and volatiles were removed by lyophilization. Remaining materials were washed with Et₂O (3×10 mL) and dried. Yield—134 mg (67%); an olive colored solid.

Biot₁-PHEAA:

To a solution of biot₁-PNPA (30 mg) in DMSO (1 mL) was added ethanolamine (100 μL), and the mixture was kept for 48 h under 70° C. Product was purified by size-exclusion chromatography, Sephadex LH-20, 0.1 M HCl MeCN/H₂O, fractions with product were evaporated and the remains was dried in vacuum. Yield—15.3 mg (93%); a white solid; ¹H NMR (D₂O, 500 MHz): 7.900-7.850 (m, 3H, standard), 4.620 (ddd, 1H, H-4 biot), 4.430 (dd, 1H, H-3 biot), 3.850-3.750 (m, 5M, H-5a biot, 2×CH₂NHCO), 3.670 (br s, 185H, CH₂NHCO PHEAA), 3.008 (dd, 1H, H-5b biot), 2.811 (dd, 1H, H-2 biot), 2.683, 2.575 (t, 2×2H, J=7.3 Hz, CH₂CO biot, AC), 2.230 (br. s, 76H CH PHEAA), 2.160-1.900 (m, 152H, CH₂ PHEAA), 1.880-1.370 (m, 170H, CH₂OH), 1.350-1.150 (m, 6H, 3×CH₂).

To determine amount of biotin in the polymer, sample containing biot₁-PHEAA (10 mg) and of 3,4-diaminobenzoic acid (152 μg, 1.0 μmol) as internal standard was prepared; ¹H NMR (D₂O, 500 MHz): 7.900-7.850 (m, 2H, H-5,6 standard), (dd, 1H, H-2 standard), 4.430 (dd, 0.52H, H-3 biot), 3.005 (dd, 0.52H, H-5b biot), (m, 0.52H, H-2 biot). According to the relative intensities of biotin and the standard signals amount of biotin in the sample was 0.52 μmol. The number of monomer units per biotinylated end group was calculated from the equation:
m=X_{biot}×(M_{biot}+kₘₒₙ×Mₘₒₙ)
where X_{biot}—the amount of biotin in the sample, M_{biot}—molecular weight of the biotinylated end group, Mₘₒₙ—molecular weight of the monomer unit, kₘₒₙ—number of monomer units per biotinylated end group; m—mass of the polymer sample.
Poly(Acrylic Acids):

To a solution of biot₁-PNPA or PNPA (30 mg) in DMSO (1 mL) was added 2M aqueous NaOH (1 mL), and the mixture was kept for 48 h under 70° C. Product was purified by size-exclusion chromatography, Sephadex LH-20, 0.1 M HCl MeCN/H₂O, fractions with product were evaporated and the remains was dried in vacuum. Yields—90%; white solids.

Biot₁-PHEAA-(Bₜᵣᵢ)₃:

To a solution of biot-PNPA (4.65 mg, 27.5 μmol) in DMSO (500 μL) were added Bₜᵣᵢ-O(CH₂)₃NH₂ (3 mg, 5.5 μmol) and NEt₃ (1.5 μL, 11 μmol). The reaction mixture was kept 12 h under 40° C., elimination of the free glycoside was confirmed by TLC (R_{f}0.57, eluent—EtOH/H₂O/Py/AcOH 3:1:1:1), ethanolamine (100 μL) was added and the reaction mixture was stayed at 40° C. for another 24 h. The product was purified by size-exclusion chromatography, Sephadex LH-20, elution—MeCN/H20 1:1. yield—5.4 mg (93%), a white solid.

Enzyme-Linked Immunosorbent Assay.

Before test plates “Reacti-Bind Streptavidin Coated High Binding Capacity Black 96-Well Plate” (Pierce) were rinsed twice with PBS. Then serial tenfold dilutions of the biotinylated glycoconjugates contained Bₜᵣᵢ in PBS (0.02-200 μg ml⁻¹, 1001 μL per well) were added onto the plates for 1 h at 37° C. Plates were washed with PBS containing 0.1% Tween-20 (washing buffer). The same way plates were washed between all the next steps. After washing the plates were blocked with 3-% BSA in PBS. B8 mAbs (1:100 in PBS containing 0.3% BSA) were added and plates were incubated for 1 h at 37° C. After that plates were washed and incubated with Ig-AP (1:5000 in PBS containing 0.3% BSA) for 1 h at 37° C. Bound antibodies were revealed with 4-methylumbellyferyl phosphate (10-4 M in carbonate buffer pH 9.6). After 30-min incubation at room temperature fluorescence intensity (355nm/460nm) was measured by “Victor²” multilabel counter (PerkinElmer). The collected data were expressed in fluorescence units. Each assay was done in duplicate, blank reaction was performed by omitting mAb. The blank reading was subtracted from the final fluorescence to provide the corrected fluorescence intensity values.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A glycopolymer configured for single point binding to a substrate.

2. The glycopolymer of claim 1, wherein the configuration for single point binding comprises a single biotin molecule coupled to the glycopolymer and wherein the substrate comprises immobilized streptavidin or a streptavidin derivative.

3. The glycopolymer of claim 1, wherein the biotin molecule is an end group of the glycopolymer.

4. The glycopolymer of claim 1, wherein the glycopolymer comprises the compound of formula 3 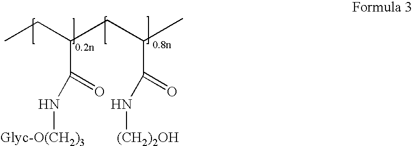
wherein n is between 30 to 10,000 and wherein Glyc comprises at least one of the carbohydrates listed in Table 1.

5. The glycopolymer of claim 1, wherein the substrate is selected from at least one of the group consisting of a bead, microsphere, slide, plate, stick, probe, array and a liposome.

6. The glycopolymer of claim 1, wherein the substrate is comprised of a material selected from the group consisting of a polymer, glass, metal and a ceramic.

7. The glycopolymer of claim 1, wherein the glycopolymer comprises at least one of the carbohydrates listed in Table 1.

8. The glycopolymer of claim 1, wherein the glycopolymer comprises a fluorescent label.

9. The glycopolymer of claim 8, wherein the fluorescent label comprises a single fluorescent label.

10. The glycopolymer of claim 1, wherein the glycopolymer comprises at least one of the group consisting of a carbohydrate-containing molecule, a macromolecule, a monosaccharide, an oligosaccharide, a polysaccharide, a glycolipid, a glycoprotein and a mimetic of a carbohydrate or carbohydrate-containing molecule.

11. A method of using the glycopolymer of claim 1, wherein the glycopolymer is used in the monitoring, diagnosing and/or prognosing of a state of health of a subject based on a subject test sample.

12. The method of claim 11, wherein monitoring, diagnosing and/or prognosing the state of health of the subject comprises-reviewing or analyzing data relating to antibodies in the subject test sample;
providing a conclusion to a patient, a health care provider or a health care manager, the conclusion being based on the review or analysis of data regarding monitoring, diagnosing and/or prognosing the state of health of the subject.

13. The method of claim 12, wherein providing a conclusion comprises transmission of the data over a network.

14. The method of claim 11, wherein the glycopolymer is used with a flow-cytometry system comprising a plurality of beads carrying the glycopolymer, using flow cytometry to detect a binding interaction between antibodies in the subject test sample and the glycopolymer, and utilizing an algorithm to identify a pattern of binding interactions previously identified as being associated with a neoplasia or risk of neoplasia.

15. The method of claim 11, wherein the glycopolymer comprises a plurality of different glycopolymers.

16. A method of synthesizing a glycoconjugate comprising a glycopolymer and a single biotin tag by ligating the compound of formula 1 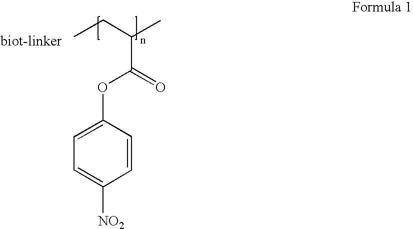
wherein n is between 30 to 10,000, with Glyc-O(CH₂)₃NH₂ (formula 2) wherein Glyc comprises at least one of the carbohydrates listed in Table 1.

17. The method of claim 16, wherein the compound of formula 1 is produced using alkycobalt(III) chelate with tridentate Schiff base coupled to biotin for initiation of polymerization of 4-nitrophenylacrylate.

18. The method of claim 16, wherein the glycopolymer comprises at least one of the carbohydrates listed in Table 1.

19. The method of claim 16, wherein the glycopolymer further comprises a single fluorescent label.

20. A glycopolymer comprising a single biotin group.

21. A compound of formula 1 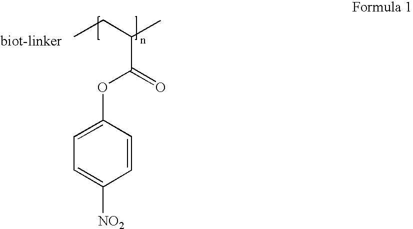
wherein n is between 30 to 10,000 and wherein biot is a single biotin.
